(19) United States
(12) Patent Application Publication
(10) Pub. No.: US 2010/0217115 A1
(43) Pub. Date: Aug. 26, 2010

(54) TEMPERATURE SENSING WITHIN A PATIENT DURING MR IMAGING

(76) Inventor: Stephen G. Hushek, Wauwatos, WI (US)

Correspondence Address: ADE & COMPANY INC., 2157 Henderson Highway, WINNIPEG, MB, R2G1P9, CA

(21) Appl. No.: 12/392,480

(22) Filed: Feb. 25, 2009

(51) Int. Cl. A61B 5/055

(52) U.S. Cl. 600/412

(57) ABSTRACT

A tear-away sheath is provided for use in an invasive procedure for insertion of a conductive device into a patient while the insertion is guided using Magnetic Resonance Imaging and has a peripheral wall surrounding a hollow interior through which a conductive device can be inserted. At least one and preferably two lines of weakness are formed along the length of the peripheral wall at which the sheath can be torn longitudinally so allow the peripheral wall to be opened to release engagement with a conductive device inserted therethrough. A temperature sensing device is embedded in the peripheral wall at or adjacent the distal end with an optical fiber extending longitudinally of the peripheral wall from the distal end to a position exposed form the patient for communicating the sensed temperature to a display device.

This invention relates to a device for sensing temperature within the body of a patient during MR imaging, which can cause elevated temperatures by RF heating of conductive structures.

### BACKGROUND OF THE INVENTION

U.S. Pat. No. 5,730,134 (Dumoulin) issued Mar. 24, 1998 to General Electric discloses the use of a system in which automatically an MRI scan is terminated or reduced in power in response to detection of a raised temperature within the body of the patient caused by RF heating of an electrode within the patient. The electrode is located within an invasive device inserted into the body and the temperature sensor is an optical thermocouple mounted also within the invasive device.

U.S. Pat. No. 5,209,233 (Holland) issued May 11, 1993 to Picker International discloses a similar system in which automatically an MRI scan is terminated in response to detection of a raised temperature at a cardiac monitoring electrode attached to the patient to prevent burning. The temperature sensor is an optical thermocouple mounted within the clip.

U.S. Pat. No. 6,185,443 (Crowley) issued Feb. 6, 2001 to Boston Scientific discloses an interventional device for minimally invasive diagnostic and therapeutic procedures where the device or probe carries sensors with a display on a distal end of the device to display to the user the sensed data including temperature.

U.S. Pat. No. 6,270,463 (Morris) issued Aug. 7, 2001 to Medrad discloses a system for measuring temperature in a strong magnetic field.

U.S. Pat. No. 6,939,327 (Hall) issued Aug. 7, 2001 to Cardiac Pacemakers discloses a peal away sheath.

The introduction of Deep Brain Stimulation (DBS) leads and other conductive structures under guidance by Magnetic Resonance Imaging is not a new clinical procedure. Various papers have been written characterizing the MR environment and analyzing how the heat is generated by the RF field applied during the MRI for implanted devices. Significant efforts have attempted to modify the resonant structure of the implant, to detect when energy is being coupled into an implanted device or otherwise to detect unsafe conditions.

Thus during MR scanning, it is well known that the high intensity electric fields created around the tips of long conductive structures inside a patient's body will create high current densities in the tissue and thus high temperatures that could burn the tissue. It can however be difficult to predict when hazardous temperatures will be created because of the variability of the electrical properties of the patient's tissue, the variability of the geometry of the antenna structures involved, the variability of the MR systems that are used and the variability of the associated equipment that is used to implant the device.

### SUMMARY OF THE INVENTION

It is one object of the invention to provide a temperature sensing apparatus for use in an invasive procedure for insertion of a conductive device into a patient while the insertion is guided using Magnetic Resonance Imaging.

According to one aspect of the invention there is provided an apparatus for use in an invasive procedure for insertion of a conductive device into a patient while the insertion is guided using Magnetic Resonance Imaging, the apparatus comprising:

an elongate sheath extending from a first end arranged to be exposed outside the body of the patient to a distal end arranged to be located within the body of the patient at a target location;

the elongate sheath having a peripheral wall surrounding a hollow interior through which a device can be inserted;

at least one line of weakness formed along the length of the peripheral wall at which the sheath can be torn longitudinally to allow the peripheral wall to be opened to release engagement with a conductive device inserted therethrough;

and a temperature sensing device located in the peripheral wall at or adjacent the distal end and having a communication medium extending longitudinally of the peripheral wall from the distal end to a position exposed from the patient for communicating the sensed temperature to a display device.

Preferably there are two lines of weakness so that the peripheral wall splits into two parts longitudinally. These can be at diagonally spaced positions. However other arrangements can be used

Preferably the two parts each have a manually engagable element in the form of a projecting handle at the first end for manually pulling the two parts apart.

Preferably the communication medium is an optical fiber which uses a fluorescent material at a position adjacent an end of the optical fiber in an arrangement known as an optical thermocouple. However other temperature sensing systems can be used although these preferably avoid the use of conductive elements which can exacerbate the heating problem.

Preferably the communication medium is embedded in the peripheral wall.

In another embodiment, instead of using an optical fiber, a regular thermocouple could be used and its sensitive region is covered with a material that is thermally conductive but an electrical insulator.

According to a second aspect of the invention there is provided a method for embedding a conductive device into patient comprising:

applying a stereotactic positioner to the body of the patient;

defining with a trajectory guide at the positioner a path for insertion of the conductive device to a target location;

inserting a sheath along the path to the target location so that the elongate sheath extends from a first end exposed outside the body of the patient to a distal end located within the body of the patient at a target location;

the elongate sheath having a peripheral wall surrounding a hollow interior through which the conductive device is inserted;

guiding the insertion using Magnetic Resonance Imaging of the target location and the conductive device within the sheath;

during the MRI, sensing data relating to the temperature of tissue within the body by a temperature sensing device located in the peripheral wall at or adjacent the distal end of the sheath and communicating the data longitudinally of the peripheral wall from the distal end to a position exposed from the patient for communicating the data to a temperature display device;

and when the conductive element is located at the target location, tearing the sheath along at least one line of weakness formed along the length of the peripheral wall to allow the peripheral wall to be opened to release engagement of the sheath with the conductive device, removing the sheath and leaving the conductive device in place.

The arrangement disclosed herein provides an optical thermocouple integrated into a peel-away sheath that measures heating at the sheath tip during the MR imaging.

As stated hereinbefore, during MR scanning, high intensity electric fields can be created around the tips of long conductive structures inside a patient's body and create high current densities in the tissue and thus high temperatures that could burn the tissue. It can be difficult to predict when hazardous temperatures will be created because of the variability of the electrical properties of the patient's tissue, the variability of the geometry of the antenna structures involved, the variability of the MR systems that could be used and the variability of the associated equipment that could be used to implant the device. One consistent aspect of the situation is that the heat is always generated at the tip of the conductive structure, so a single point measurement will provide verification of the safety of the procedure.

The rate of temperature increase is proportional to the amount of RF power used and can also vary widely. Displaying the detected temperature allows a physician or other staff member to stop imaging if excessive temperatures are reached. All MR scanners allow for scan interruption as a patient safety feature for patients who suddenly feel claustrophobic, feel ill or otherwise require interruption of the scan.

The system disclosed herein is preferably arranged to merely display the detected temperature and to rely on the discretion of the physician or staff member to decide if/when to terminate the scan.

The invention has been described in the context of DBS lead implantation but it is also relevant to and can be used with the introduction of any invasive conducting structure inserted within the body during MR imaging.

### BRIEF DESCRIPTION OF THE DRAWINGS

One embodiment of the invention will now be described in conjunction with the accompanying drawings in which:

FIG. 1 is a schematic illustration of a sheath according to the present invention and to a method of use of the sheath.

FIG. 2 is an isometric view of an outer end of the sheath of FIG. 1.

FIG. 3 is an isometric view of a distal end of the sheath of FIG. 1.

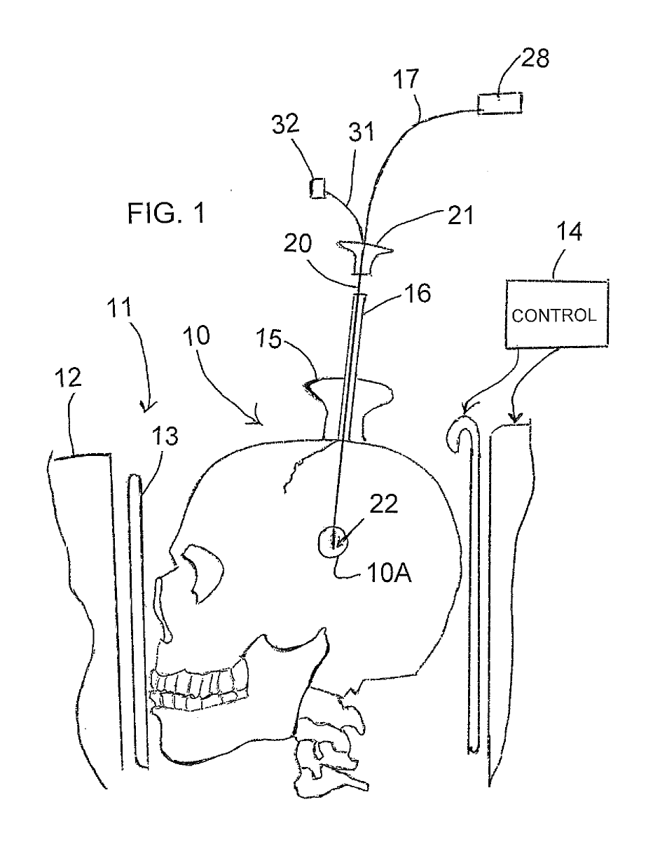

In the drawings like characters of reference indicate corresponding parts in the different figures.

### DETAILED DESCRIPTION

The apparatus of the present invention is mainly shown only schematically as many of the components are well known to persons skilled in this art. In particular there is shown a part of the patient shown at 10 which is located during at least a part of the procedure in an MR imaging system 11 including a magnet 12, RF coils 13 and a control and display system 14 for use in an invasive procedure for insertion of a conductive device into a patient while the insertion is guided using Magnetic Resonance Imaging. The conventional components further include a stereotactic positioner 15 and a cannula 16 located by the positioner 15 so as to define a path for insertion of a conductive device to a target location.

The conventional components further include a conductive electrode 17 which in regard to DBS can be either a micro-recording electrode that is temporarily positioned at the target or a stimulation electrode intended to be located at and remain in position at a target location in the brain of the patient.

The arrangement of the present invention comprises an elongate sheath 20 extending from a first end 21 exposed outside the body of the patient to a distal end 22 located within the body of the patient 10 at a target location 10A.

The elongate sheath 20 is formed from a plastics material well known for such invasive procedures and has a wall thickness of the order of 50 microns to 500 microns with an outside diameter of 1 mm to 3 mm and an interior hollow bore of the order of 0.5 mm to 2.5 mm diameter. The size is adjusted so that the electrode 17 operates as a sliding fit.

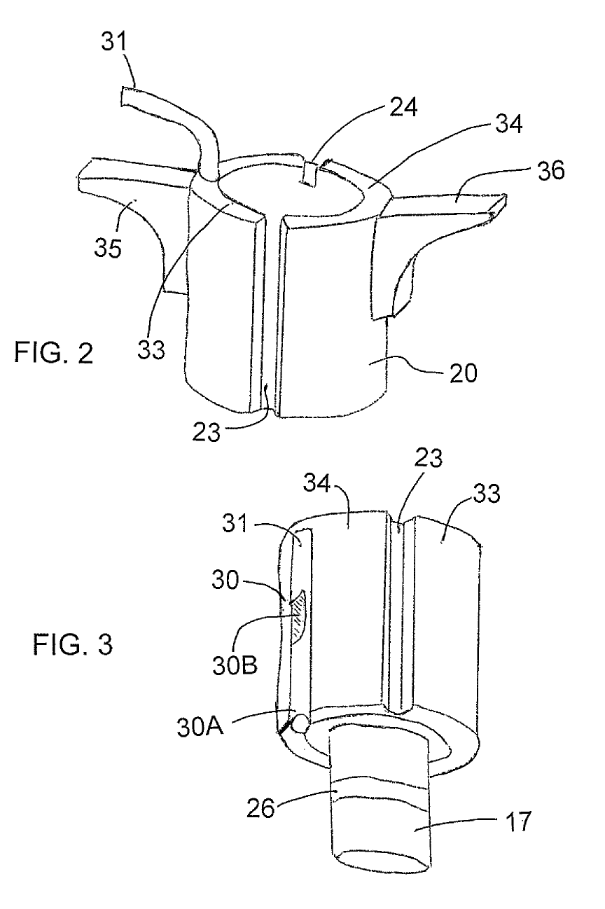

The two ends of the sheath are shown in FIGS. 2 and 3. The sheath is molded with at least one line of weakness and as shown there are two thinner lines or lines of weakness in the wall as shown at 23 and 24, each extending along the full length of the sheath. The thin lines 23 and 24 are formed along the length of the peripheral wall at which the sheath can be torn longitudinally to allow the peripheral wall to be opened to release engagement with the conductive electrode inserted therethrough.

As shown in FIG. 3, the distal end of the stimulation electrode 17 includes a contact 26 for communication with surrounding tissue in the brain. Typical DBS stimulating electrodes contain 5 contacts, the other 4 of which are inside the sheath and not visible in FIG. 3. The proximal end of the electrode contains a connector 28, which connects to a lead extension that is tunneled under the skin to a control device (not shown). The control device is located in a pocket created near the patient's clavicle (collar bone) after the installation is complete.

A temperature sensing device 30 is located embedded in the peripheral wall at or adjacent the distal end and has a communication medium in the form of an optical fiber 31 extending longitudinally of the peripheral wall from the distal end to a position exposed from the patient for communicating the sensed temperature to a control and display device shown schematically at 32.

The two parts 33 and 34 of the sheath defined on each side of the lines of weakness 23 and 24 each have a manually engagable handle 35 and 36 at the first end projecting outwardly to the side of the sheath for manually pulling the two parts apart.

As previously known, the temperature sensing device 30 includes a fluorescent material forming a sensor at a position adjacent to but spaced from an end 30A of the optical fiber. The sensor in the optical fiber defined by the fluorescent material is exposed to the tissue at a location 30B adjacent to but spaced from the end 30A of the sheath. The optical fiber is otherwise fully enclosed by the sheath material. The fluorescent material has a temperature dependent response allowing the control device 32 to generate optical signals which are transmitted and returned through the fiber with the return signal being dependent upon and indicative of the temperature.

In FIG. 3 the end 30A of the fiber is located at the end of the sheath with the sensor 30 spaced from the end. However the fiber may project beyond the end of the sheath so that the sensor portion 30B is located at or slightly beyond the end of the sheath to allow detection of the temperature of the tissue just beyond or directly at the end of the sheath where the temperature rise generated by the conductor is most concentrated.

In the method for embedding a conductive device into a patient the following steps are applied:

the stereotactic positioner is attached to the body of the patient and adjusted until its trajectory guide is oriented along the desired trajectory;

the sheath with a stylet inserted therein is fed through the trajectory guide along the desired trajectory until it reaches the target location, using Magnetic Resonance Imaging of the target location and the trajectory;

the stylet is removed and replaced with the electrode;

during the MRI, data relating to the temperature of tissue within the body is obtained by the temperature sensing device and communicated to the temperature display device;

when imaging has confirmed that the conductive element is indeed located at the target location, the conductive element is fixed in place and the sheath is torn along the lines of weakness to allow the peripheral wall to be opened to release engagement of the sheath with the conductive device allowing the sheath to be split and removed by pulling on the handles to pull the sheath parts out of the body leaving the conductor in place.

The active temperature sensing region of the optical thermocouple is approximately 0.5 m m back from the tip of the optical fiber. The sheath wall thickness is in one example approximately 100 microns The optical fiber can also protrude forward from the sheath so that the temperature sensing region is at the tip of the sheath. The configuration would be desirable for some clinical purposes because the distance between the tip of the conductive element and the sensitive region of the sensor would be reduced. If the high temperature region is extremely localized this configuration may be required. The configuration where the entire optical fiber is encased within the sheath is also a desirable configuration because the minimal amount of tissue is disturbed.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. Apparatus for use in an invasive procedure for insertion of a conductive device into a patient while the insertion is guided using Magnetic Resonance Imaging, the apparatus comprising:
an elongate sheath extending from a first end arranged to be exposed outside the body of the patient to a distal end arranged to be located within the body of the patient at a target location;
the elongate sheath having a peripheral wall surrounding a hollow interior through which a device can be inserted;
at least one line of weakness formed along the length of the peripheral wall at which the sheath can be torn longitudinally to allow the peripheral wall to be opened to release engagement with a device inserted therethrough;
and a temperature sensing device located in the peripheral wall at or adjacent the distal end and having a communication medium extending longitudinally of the peripheral wall from the distal end to a position exposed from the patient for communicating the sensed temperature to a display device.

2. The apparatus according to claim 1 wherein there are two lines of weakness so that the peripheral wall splits into two parts longitudinally.

3. The apparatus according to claim 1 wherein the two parts each have a manually engagable element at the first end for manually pulling the two parts apart.

4. The apparatus according to claim 1 wherein the manually engagable element comprises a projecting handle.

5. The apparatus according to claim 1 wherein the communication medium is an optical fiber.

6. The apparatus according to claim 5 wherein the temperature sensing device includes a fluorescent material at a position adjacent an end of the optical fiber.

7. The apparatus according to claim 6 wherein the temperature sensing device comprises an optical thermocouple.

8. The apparatus according to claim 1 wherein the communication medium is embedded in the peripheral wall.

9. A method for embedding a conductive device into patient comprising:
applying a stereotactic positioner to the body of the patient so as to define with a trajectory guide thereof a desired trajectory to a target location;
inserting a sheath along the path to the target location so that the elongate sheath extends from a first end exposed outside the body of the patient to a distal end located within the body of the patient at a target location;
the elongate sheath having a peripheral wall surrounding a hollow interior;
guiding the insertion using Magnetic Resonance Imaging of the target location and the sheath;
during the MRI, sensing data relating to the temperature of tissue within the body by a temperature sensing device located in the peripheral wall at or adjacent the distal end of the sheath and communicating the data longitudinally of the peripheral wall from the distal end to a position exposed from the patient for communicating the data to a temperature display device;
inserting a conductive element through the sheath so that an end of the conductive device is located at the target location;
and tearing the sheath along at least one line of weakness formed along the length of the peripheral wall so allow the peripheral wall to be opened to release engagement of the sheath with the conductive device and removing the sheath.

10. The method according to claim 9 wherein there are two lines of weakness so that the peripheral wall splits into two parts longitudinally.

11. The method according to claim 9 wherein the two parts each have a manually engagable element at the first end for manually pulling the two parts apart.

12. The method according to claim 9 wherein the manually engagable element comprises a projecting handle.

13. The method according to claim 9 wherein the communication medium is an optical fiber.

14. The method according to claim 13 wherein the temperature sensing device includes a fluorescent material at a position adjacent an end of the optical fiber.

15. The method according to claim 14 wherein the temperature sensing device comprises an optical thermocouple.

16. The method according to claim 9 wherein the communication medium is embedded in the peripheral wall.
